(19) United States
(12) Patent Application Publication
(10) Pub. No.: US 2009/0155835 A1
(43) Pub. Date: Jun. 18, 2009

(54) DIAGNOSTIC METHOD AND PROGNOSTIC TOOL FOR RHEUMATOID ARTHRITIS

(76) Inventors: Morris Frank Manolson, Toronto (CA); Svetlana Komarova, Kirkland (CA); Rene Harrison, Toronto (CA); Artur De Brum Fernandes, Sherbrooke (CA); Gilles Boire, Sherbrooke (CA); Lukasz Kurgan, Edmonton (CA); S. Jeffrey Dixon, London (CA); Stephen M. Sims, London (CA)

Correspondence Address: DOBRUSIN & THENNISCH PC, 29 W LAWRENCE ST, SUITE 210, PONTIAC, MI, 48342, US

(21) Appl. No.: 12/250,896

(22) Filed: Oct. 14, 2008

Related U.S. Application Data
(60) Provisional application No. 60/960,749, filed on Oct. 11, 2007.

(51) Int. Cl. C12Q 1/02

(52) U.S. Cl. 435/29

(57) ABSTRACT

The invention relates to a diagnostic method and prognostic tool for rheumatoid arthritis and uses thereof.

### CROSS-REFERENCE TO RELATED APPLICATIONS

This application claims the benefit of the filing date of U.S. Provisional Application Ser. No. 60/960,749, filed Oct. 11, 2007 and entitled “Diagnostic Method and Prognostic Tool For Rheumatoid Arthritis” and which is herein incorporated by reference.

### FIELD OF THE INVENTION

The invention relates to a diagnostic method and prognostic tool for rheumatoid arthritis and uses thereof.

### BACKGROUND OF THE INVENTION

Bone is a central element in the pathophysiology of degenerative and inflammatory arthropathies. Rheumatoid arthritis (RA) is associated with periarticular bone loss and erosions which contribute to joint destruction.

It is estimated that 1% of the population currently suffer from RA (about 70 million people). The degree of severity and progression varies greatly.

Current treatment of RA includes anti-inflammatory and remission-inducing drugs such as anti-malarials, methotrexate and the newer biological therapies based on antibodies directed against certain elements of the immune system.

Early treatment of RA has been shown to be extremely important to control the disease and to prevent joint destruction and the ensuing disability (Wiles N J, et al., “Reduced disability at five years with early treatment of inflammatory polyarthritis: results from a large observational cohort, using propensity models to adjust for disease severity”, Arthritis Rheum. 2001; 44:1033-42). The very early diagnosis and prognosis of rheumatoid arthritis is presently difficult to attain. The diagnosis of RA depends both on clinical evaluation and laboratory tests such as rheumatoid factor and anti-CCP antibodies, as well as genetic markers. Their value is however limited and they must be used in conjunction with clinical and radiologic evaluation.

Many models have been proposed to establish prognosis in early rheumatoid arthritis. One of the most popular relies on a combination of clinical, serological and radiologic variables to predict persistence of arthritis and development of erosions (Visser H, le Cessie S, Vos K, Breedveld F C, Hazes J M., “How to diagnose rheumatoid arthritis early: a prediction model for persistent (erosive) arthritis”, Arthritis Rheum, 2002; 46:357-65). There is no single test to diagnose the disease. Although some serologic markers exist that may contribute to the diagnosis and prognosis (Boire G, et al., “Anti-Sa antibodies and antibodies against cyclic citrullinated peptide are not equivalent as predictors of severe outcomes in patients with recent-onset polyarthritis”, Arthritis Res Ther. 2005; 7(3):R592-603), it is often hard, in the first months of evolution, to make a definite diagnosis of RA and to determine the risk of aggressive disease. This point is very important considering the concept of a therapeutic window in early arthritis, during which aggressive treatment of severe disease would be more effective (Bejarano V, et al, “Yorkshire Early Arthritis Register Consortium. Effect of the early use of the anti-tumor necrosis factor adalimumab on the prevention of job loss in patients with early rheumatoid arthritis”, Arthritis Rheum. 2008 Sep. 29; 59(10):1467-1474). Given the risks and the cost of these treatments, being able to detect patients with severe disease early during its course and in one embodiment, monitoring their disease state and progression during treatment, would allow for the development or selection of more effective treatment regimes for patients, such as targeting them for more aggressive therapy. This would help to decrease long term disability and health care costs for said patient population.

As such, there is a need for a diagnostic method for RA and the ability to determine and/or predict the (1) activity and (2) severity of RA. The development of a prognostic tool to allow for prediction of progression of disease and a method that would enable the monitoring of same over time or throughout a treatment regime in a patient would also be useful. Thus would assist in the clinical management and/or treatment of patients and to tailor treatment regimes for them.

### SUMMARY OF THE INVENTION

The present invention relates to a method for the diagnosis and/or prognosis of rheumatoid arthritis (RA) in a subject comprising assaying a sample from the subject and measuring: (a) numbers of osteoclasts generated from peripheral blood mononuclear cells, (2) apoptosis of said generated osteoclasts, and/or (3) resorptive activity of said osteoclasts wherein a change in any such values as compared to a control sample is useful in the diagnosis and/or prognosis of RA and/or disease state, including activity. In one embodiment an increase in number of osteoclasts and/or decrease in osteoclast apoptosis and/or an increase in osteoclast resorptive activity compared to a control sample, in one embodiment a control sample of normal or non-RA subjects, is indicative of RA. In one embodiment the values predict the activity and/or severity and/or aggressiveness of RA. Surrogate markers of the number of osteoclasts, osteoclast apoptosis or osteoclast resorption activity can be used for the same purposes.

The invention also relates to a method of screening a subject for RA comprising: (a) obtaining a biological sample from a subject, in one embodiment a sample comprising osteoclast precursors, such as human peripheral blood mononuclear cells; (b) detecting the number of osteoclasts and/or amount of osteoclast apoptosis and/or osteoclast resorptive activity in said sample; and (c) comparing said values of osteoclasts and/or amount of osteoclast apoptosis and/or osteoclast resorptive activity detected to a predetermined normal, where detection of an increase in osteoclast numbers and/or a decrease in osteoclast apoptosis and/or an increase in osteoclast resorptive activity compared to a control sample is indicative of the presence, prognosis, aggressiveness, activity and/or severity of rheumatoid arthritis.

In one embodiment, the “sample” or “biological sample”, is any material known to or suspected of expressing or containing osteoclasts, its precursors or surrogate markers of the presence, differentiation capacity, activity or apoptosis of these cells. The test sample can be used directly as obtained from the source or following a pretreatment to modify the character of the sample. In one embodiment, the biological sample is a blood sample or serum or tissue extracts. In one embodiment it is serum or a fraction thereof, or any sample comprising an osteoclast precursor, such as peripheral blood mononuclear cells (PBMCs).

The term “subject” refers to a warm-blooded animal such as a mammal which is afflicted with or suspected to be afflicted with rheumatoid arthritis. Preferably, “subject” refers to a human.

The invention also relates to kits for carrying out the methods of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1A is a bar graph illustrating the number of osteoclasts per well in control versus RA subjects (RA (total), RA (act) and RA (inact) derived from PBMC samples from subjects) as described in Example 1.

FIG. 1B is a bar graph illustrating the number of osteoclasts/well divided by the number of CD 14+ cells (osteoclast precursors) in the blood sample studied in control versus RA subjects (RA (total), RA (act) and RA (inact) derived from PBMC samples from subjects) as described in Example 1.

FIG. 2 is a bar graph illustrating the percentage of osteoclasts in apoptosis after the cells were kept for 24 h without M-CSF and RANKL in 5% FBS in controls (non-RA subjects) versus RA subjects (RA (total), RA (act) and RA (inact) derived from PBMC samples from subjects) as described in Example 2.

FIG. 3 is a bar graph illustrating the resorption activity of osteoclasts in control (non-RA subjects) versus RA subjects (RA (total), RA (act) and RA (inact) derived from PBMC samples from subjects) as described in Example 3.

FIG. 4 is a bar graph illustrating the mean number of osteoclasts derived from PBMC sample of subjects with severe RA (>/=1.5) versus less severe RA (</=1.5) as described in Example 4.

FIG. 5 illustrates the OC distribution of the number of OCs/well of self-reported healthy individuals. Horizontal bars represent the average±standard deviation (n=91).

### DETAILED DESCRIPTION OF THE INVENTION

Rheumatoid arthritis (RA) is associated with periarticular bone loss and erosions which contribute to joint destruction to which osteoclasts (OC) are critical. Although the involvement of OC in bone and joint destruction in RA has been confirmed, their role in disease onset and progression was previously not known. The present inventors have now surprisingly shown that the capacity to generate OC from PBMC in culture, the degree of OC apoptosis and resorption activity can independently or in combination be used as diagnostic and/or prognostic indicators of RA. Further, the present inventors herein show that OC values can also be used as predictors of severity of RA onset and/or diagnostic indicators of RA activity. In one embodiment, the inventors have shown that OC values derived from culturing Human peripheral blood mononuclear cells (PBMCs) taken from blood samples from patients and their subsequent degree of apoptosis of said OCs and/or resorption activity can be used as diagnostic indicators of RA and severity and activity of same.

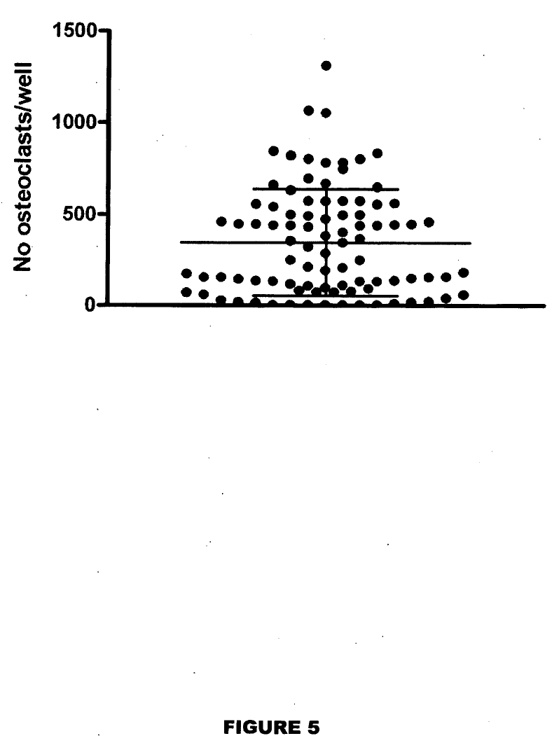

Human peripheral blood mononuclear cells (PBMCs) can differentiate into OC capable of bone resorption. The capacity for in vitro osteoclastogenesis varies widely in a normal human population, distinguishing two subgroups, high and low differentiators, regardless of age, gender, weight, or other demographic variables. FIG. 5 illustrates the osteoclast distribution in a normal human population.

In one embodiment, the present inventors have herein shown that the capacity to generate OC in vitro from peripheral blood mononuclear cells correlates with the diagnosis of RA and that this parameter or any other parameter derived from it and being able to predict the osteoclastogenic capacity of an individual could be used for the early diagnosis and prognosis of RA. Such information would be useful in the development of a treatment regime for a subject in need thereof and/or to optimize a treatment regime in a subject, in one embodiment, for monitoring the effectiveness of same by monitoring changes in OC, apoptosis and/or resorption in samples of a subject over time or as compared to internal and/or external controls. Said controls including but not limited to subjects or a population of subjects with RA on different treatment regimes or no treatment and/or subjects without RA who are subject to the same or no treatment regime. For instance, wherein an increase in or higher value of OC, resorption and/or a decrease in apoptosis as compared to a control (e.g. prior values of said subject or others with RA on no or other treatment regimes or to values of a normal subject or population (without RA)), can be indicative of a not as an effective treatment for said subject as compared to a control, whereas the lower values in a treatment population versus a non-treatment or other treatment population (with RA or without RA) or lowering of values of OC, resorption and/or an increase in apoptosis as compared to said controls can be indicative of a more effective treatment regime and/or amelioration of a disease state in said subject.

RA, herein, was diagnosed according to the ACR Criteria for RA (Arnett F C, Edworthy S M, Bloch D A, McShane D J, Fries J F, Cooper N S, Healey L A, Kaplan S R, Liang M H, Luthra H S, et al: The American Rheumatism Association 1987 revised criteria for the classification of rheumatoid arthritis. Arthritis Rheum 31:315-24, 1988). Disease activity was defined according to the DAS28 criteria (Balsa A, Carmona L, Gonzalez-Alvaro I, Belmonte M A, Tena X, Sanmarti R: Value of Disease Activity Score 28 (DAS28) and DAS28-3 compared to American College of Rheumatology-defined remission in rheumatoid arthritis. J Rheumatol 31:40-6, 2004; Fransen J, Creemers M C, Van Riel P L: Remission in rheumatoid arthritis: agreement of the disease activity score (DAS28) with the ARA preliminary remission criteria. Rheumatology (Oxford) 43:1252-5, 2004).

In one embodiment, the invention provides a method for diagnosing RA in a subject and/or determining prognosis of RA in a subject, including, in one embodiment but not necessarily limited to determining the activity and/or severity of RA in a subject comprising:

(a) obtaining a sample from the subject. In one embodiment the sample is a sample comprising OC precursors, such as PBMCs capable of differentiating to OCs. In one embodiment the sample is a blood and/or serum sample. In one embodiment a minimum of 50 mls is obtained. In one embodiment, the OC precursors, in one embodiment, PBMCs are isolated from the sample to the extent necessary to enable the differentiation of the OC precursors, such as PBMCs to OC. In one embodiment PBMCs are isolated from 50 ml of blood by Ficoll-Hypaque gradient. In one embodiment, the isolated OC precursors, such as PBMCs are cultured under conditions that promote differentiation into OCs, such as described in Example 1, however persons of skill in the art would be familiar with other conditions. In one embodiment, the whole population of PBMCs is plated in 48-well tissue culture plates containing a bone slice or a glass slide, and the cells are allowed to differentiate for 21 days in the presence of recombinant RANKL (75 ng/ml) and M-CSF (10 ng/ml). However the length of culturing (e.g. time, days) or other culturing conditions can vary depending on the amount of OCs desired. Controls should be cultured under the same conditions or values standardized taking into account any variations of culturing conditions; and

(b) measuring and/or determining one or more of the following:

(i) amount of OCs derived from the sample, OC precursors or PBMC. This can be determined as described in the Examples although other methods known in the art could be used. In one embodiment, after 21 days in culture the cells are fixed and stained for TRAP activity and with hematoxylin. The number of TRAP+cells containing three or more nuclei per well in a 48 wells plate is counted in each well. (Durand M, Gallant M A, de Brum-Femandes A J. Prostaglandin D2 receptors control osteoclastogenesis and the activity of human osteoclasts. J Bone Miner Res. 2008 July; 23(7):1097-10) and are indicative of the amount of OCs derived from PBMCs;

(ii) level of OC apoptosis. Again, various methods may be known in the art. In one embodiment level of apoptosis can be determined as described in Example 2. In one embodiment, OCs differentiated with M-CSF and RANKL for 21 days are then kept for 24 h without M-CSF and RANKL in 5% FBS. Cell death is visualized using the TACS Blue kit from R&D Systems;

(iii) level of OC resorption activity. Various methods may be known in the art. In one embodiment level of resorption activity can be determined as described in Example 3. In one embodiment, for bone resorption assays, cells differentiated for 21 days on bone slices are stained for TRAP and 0.2% toluidine blue. Resorption surface area is quantified using the image analysis program, Simple PCI. (Durand M, Gallant M A, de Brum-Fernandes AJ. Prostaglandin D2 receptors control osteoclastogenesis and the activity of human osteoclasts. J Bone Miner Res. 2008 July; 23(7):1097-10); and

(c) determining whether the subject has RA, the prognosis of RA, the severity of RA and/or disease activity, wherein any changes in the amount of OC, apoptosis or resorptive activity as compared to a control can be useful in the diagnosis, including prognosis, severity and/or activity of RA.

In one embodiment:

(i) the amount of OC is different than a control, in one embodiment the amount of OC, such as, in RA inactive subjects is higher than a control;

(ii) the level of OC apoptosis is lower than a control; and/or

(iii) the level of OC resorption is higher than a controls

is indicative of the subject having RA, and/or is indicative of a more active and/or severe and/or aggressive disease state than the control. In one embodiment the control can be a control as described herein. In one embodiment, the control are one or more values obtained from a healthy population of subjects or in one embodiment the mean or normal range of values obtained from said control population.

In one embodiment, a control sample may correspond to numbers of OC, osteoclast apoptosis, and/or osteoclast resorptive activity quantitated for samples from healthy, non-RA control subjects, from a random sample of the general population, from samples of subjects with other known RA states (e.g. active, non-active RA or mean or averaged values of all subjects in said selected population with RA or from internal controls using other assayed samples of the subject. In one embodiment the methods of the present invention enable the generation of OC, apoptosis and resorptive values in a number of RA disease states that can establish value ranges for said states (such as the presence, prognosis, severity and/or activity of RA) that are standardized taken into account the methods in which the values are obtained (e.g. methods for culturing PBMCs under differentiating conditions), that can be used as comparators to determine RA, RA activity, severity and/or prognosis, by comparing the values obtained from the subject with values obtained previously or simultaneously for others with known disease states, wherein similar values will be indicative of a similar disease state for said subject. It is submitted that a person of skill in the art would appreciate what type of control could be used depending on the purpose of conducting the methods of this invention, whether, for example, it be for initial or subsequent diagnosis of disease state in a person or for monitoring the disease state of a subject over time. For instance, if one wishes to monitor the progression or effectiveness of a treatment in a subject with RA, results from prior samples of the subject could be used as comparators, where a change in values can be indicative of a change of disease state (presence, prognosis, severity and/or activity). In one embodiment, an increase in OC, resorption and/or a decrease in apoptosis could be indicative of increased severity and/or activity of RA, whereas a decrease in OC resorption and/or an increase in apoptosis could be indicative of amelioration of the disease state. So in one embodiment, the invention provides a method to monitor and determine the effectiveness of treatment regimes and/or to optimize treatment for a subject. In one embodiment the control can be values for OC, apoptosis and resorption derived from a normal health subject or population (without RA) or potentially from a randomized sample of the population (with a known percentage of RA in said population), but in one embodiment from a health subject or healthy subject population (i.e., without RA). Wherein differences in values or degree of difference from normal or control values can be used to diagnose, predict RA disease state of a subject, including prognosis, activity and/or severity of RA in said subject. The values for controls should be taken under the same or similar conditions as those taken for subject samples and/or standardized to enable comparatives. For instance if a PBMC sample is cultured under differentiating conditions to produce OC for 21 days in a subject sample, the control values should be obtained under similar conditions or the values between the subject sample and control values standardized, as required, to enable comparison for diagnostic and/or prognostic purposes.

The terms “sample”, “biological sample”, and the like mean a material known to or suspected of expressing or containing OC, OC precursors or markers of the presence of OC, differentiation capacity, activity or apoptosis of these cells. The test sample can be used directly as obtained from the source or following a pretreatment to modify the character of the sample. The sample can be derived from any biological source, such as tissues or extracts, including cells and physiological fluids, such as, for example, whole blood, plasma, serum, saliva, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins, DNA, and RNA may be isolated from the samples and utilized in the methods of the invention. In a preferred embodiment, the biological sample is serum or tissue extracts, most preferably serum or a fraction thereof, more peripheral blood mononuclear cells (PBMCs).

In one embodiment, the “subject” is a warm-blooded animal such as a mammal which is afflicted with or suspected to be afflicted with RA. Preferably, “subject” refers to a human.

In one embodiment the invention provides a method for diagnosing and/or predicting the degree of severity of RA in a subject comprising steps (a) to (b) above and then determining the number of OCs as compared to a control wherein an elevated number of OC correlates with increased severity of RA.

In another embodiment, the invention provides a method for monitoring the progression of RA comprising conducting steps (a) to (c) above and comparing the values and/or results obtained with those previously obtained from the subject to monitor any changes in values of said indicators over time.

The methods and information derived from the methods of the invention can be used to determine and/or monitor a treatment regime for a patient and the effectiveness thereof. It can also be used to determine the prognosis, severity, activity and presence of RA in a subject. It can also be used to develop a database of values (mean, average, or range of values) of OC, apoptosis and resorption associated with a particular RA disease state. In one embodiment such a database could be used for diagnostic, prognostic and/or evaluative purposes as outlined herein. Further, in one embodiment the invention provides a use of the methods of the invention for treating RA by determining the RA disease state of a subject (such as, aggressiveness, prognosis, severity and/or activity) and then treating the subject in a manner appropriate for said disease state. In one embodiment, the progress and/or effectiveness of the treatment can be monitored using the methods of the present invention and the treatment adjusted as may be required to optimize treatment of a subject with RA.

The invention also relates to kits that can be used for carrying out all or part of the methods of the invention. In one embodiment, the kits can comprise one or more of the following: material for culturing osteoclast precursors from PBMCs (e.g., Ficoll-Hypaque gradient for PBMC isolation, tissue culture plates and cell culture media) and/or material for inducing OC differentiation (ie: RANKL, M-CSF) and/or material for evaluating the number of osteoclasts formed (reagents for Tartrate Resistant Acid Phosphatase staining), osteoclast apoptosis and bone resorption by osteoclasts. In one embodiment, the kit can comprise instructions for their use and/or for conducting the methods of the present invention, such as the diagnostic and/or prognostic methods of the invention as described herein, including but not limited to a method for determining the activity and severity of RA in a subject.

The present invention will be further understood from the following non-limiting examples:

### EXAMPLES

### Example 1

### Osteoclast (OC) Generation and Capacity

One hundred and eighty (180) subjects were enrolled in the study according to applicable laws. Patients satisfying the ACR Criteria for RA (Arnett, F C, et al, “The American Rheumatism Association 1987 Revised Criteria for the Classification of Rheumatoid Arthritis”, Arthritis Rheum 1988; 31:315-24) were recruited from the outpatient rheumatology clinic at the Centre Hospitalier Universitaire de Sherbrooke, in Sherbrooke Quebec. Control subjects were recruited from the local Sherbrooke, Quebec population. A summary of the demographics of the subjects enrolled in the studies described herein can be found at Table 1.

Blood samples were taken from the subjects. PBMCs were isolated from 50 ml of blood by Ficoll-Hypaque gradient (Boyum, Scand J Clin Lab Invest Suppl 1968; 97:77-89) and the number of CD14+ osteoclast precursors was determined by FACS (Fluorescence activated cell sorter.

The whole population of PBMCs were cultured under differentiating conditions for 21 days in the presence of recombinant RANKL (75 ng/ml) and M-CSF (10 ng/ml) fixed, stained for TRAP (Tartrate Resistant Acid Phosphatase 5b) activity and for hematoxylin. The number of TRAP+cells containing three or more nuclei-were counted in each well (Durand M, Gallant M A, de Brum-Femandes A J., “Prostaglandin D2 receptors control osteoclastogenesis and the activity of human osteoclasts”, J Bone Miner Res. 2008 July; 23 (7): 1097-10) Triplicates were used for each patient.

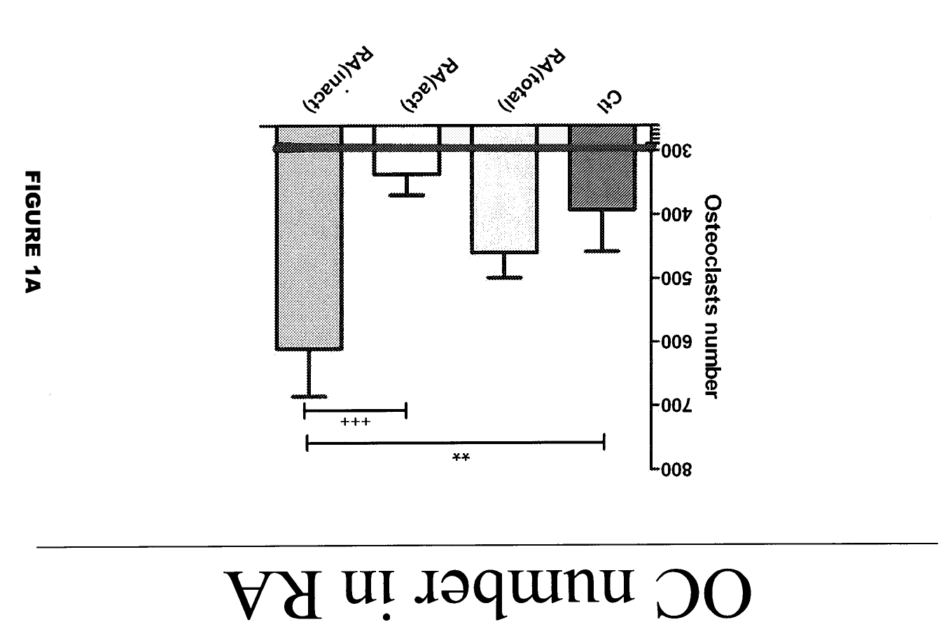

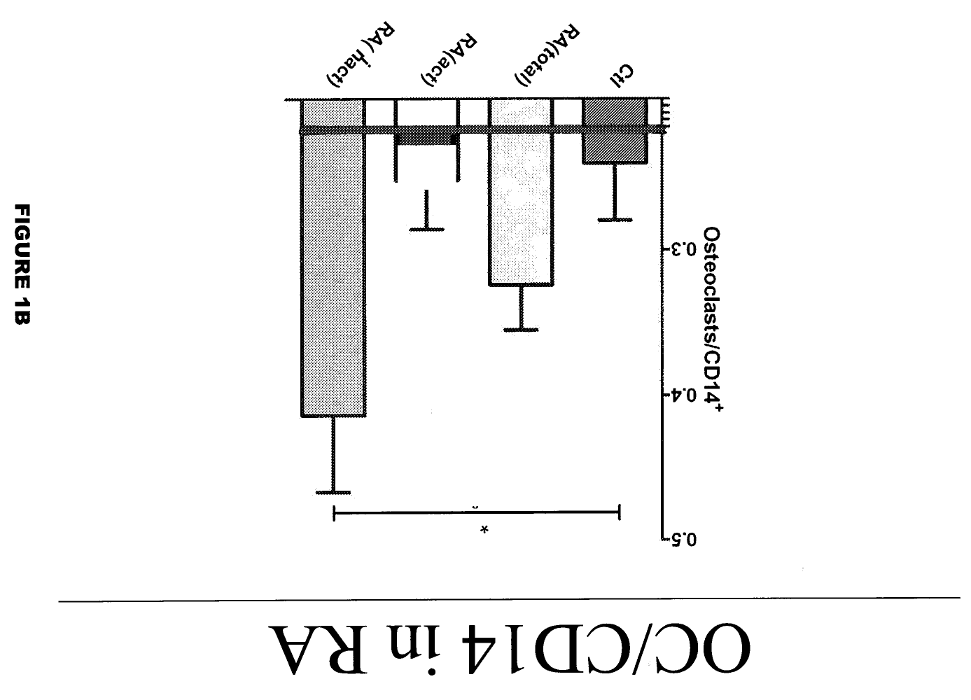

The studies demonstrate that for normal donors (n=41) the number of OCs/well was 393±65 (average±s.e.m.) TRAP+, multinuclear cells/well, while in subjects with diagnosed rheumatoid arthritis (RA) (n=139) the number of OCs/well was 459±39 TRAP+, multinuclear cells/well (see FIG. 1A). Despite having higher number of osteoclasts than the normal population studied, the number of the OC precursor CD14+ cells in the group with RA (1842±77) did not differ significantly from the control group (1917±132). FIG. 1B shows that the ratio OC generated/OC precursors is higher in subjects with RA than in controls, especially in the subgroup with inactive disease (RA (inact)), showing that these parameters may also be used, alone or in combination, to characterize disease activity. In a comparison between normal samples (n=24) and patients diagnosed with rheumatoid arthritis (RA) (n=77), numbers for CD14+ cells/10,000 cells were 1806±850 versus 1819±897, respectively.

Results are illustrated in FIGS. 1A and B where FIG. 1A is a bar graph illustrating the number of osteoclasts per well in control versus RA (total), RA (act) and RA (inact) derived from PBMC samples from the subject population described in Table 1 and cultured under conditions to promote differentiation of PBMCs to osteoclasts. RA (total) being patients diagnosed with RA with both active (act) and inactive (inact) RA. RA (act) are patients with RA with active disease, while RA (inact) are patients with RA with inactive disease defined as a DAS28>2.6 (RA (act)) or <=2.6 (RA (inact)), respectively.

FIG. 1B is a bar graph illustrating the number of osteoclasts/well divided by the number of CD 14+ cells (osteoclast precursors) in the blood sample studied in control versus RA (total), RA (act) and RA (inact) derived from PBMC samples from the subject population described in Table 1 and cultured under conditions to promote differentiation of PBMCs to osteoclasts. RA (total) are patients diagnosed with RA with both active (act) and inactive (inact) RA. RA (act) are patients with RA with active disease, while RA (inact) are patients with RA with inactive disease. The results confirm the results illustrated in FIG. 1A.

### Example 2

### OC Apoptosis

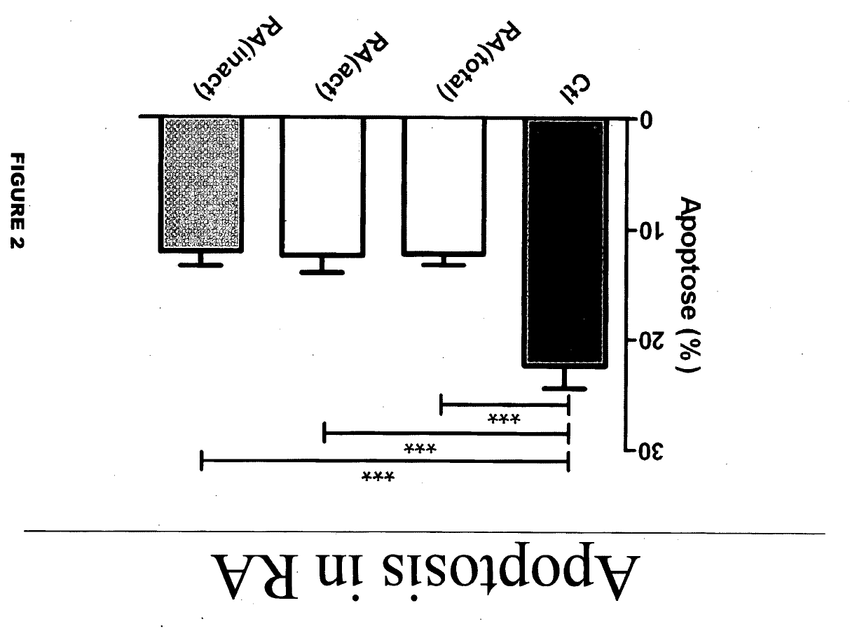

To study apoptosis, OCs were induced to differentiate from PBMC in culture for 21 days, as described above, then kept for 24 h without M-CSF or RANKL in 5% FBS. Cell death is visualized using the TACS Blue kit from R&D Systems and the percentage of OCs in apoptosis was recorded. FIG. 2 shows that, in the group of patients with RA (n=139), whether the disease is active (act) or inactive (inact), the percentage of OCs in apoptosis is significantly lower than that found in the control group (n=41).

### Example 3

### OC Resorptive Activity

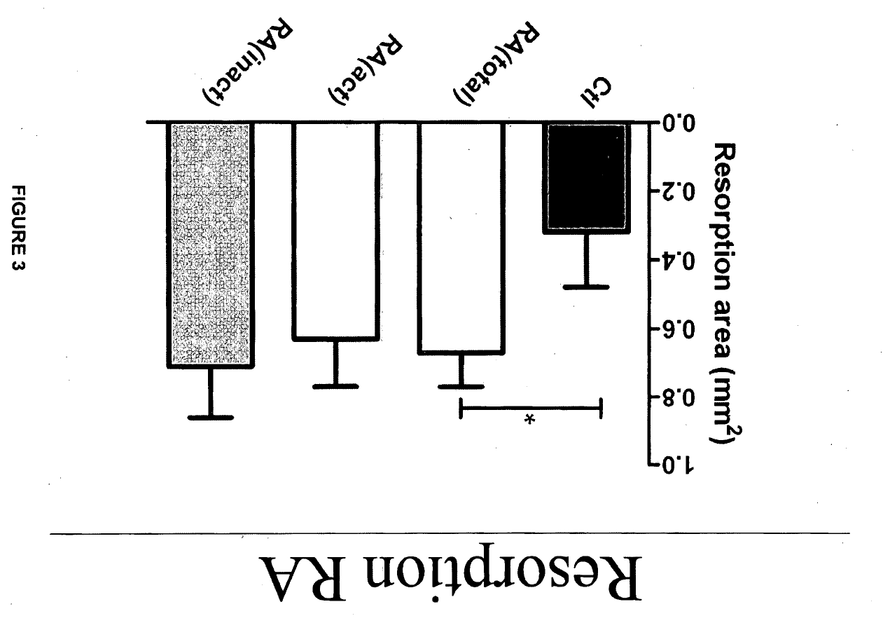

To directly assess OC resorptive activity, cells were differentiated for 21 days on bone slices, and stained for TRAP to assess OC number as described in Example 1 (above). After OC removal, bone slices were stained with 0.2% toluidine blue, and the total area of bone was quantified as described elsewhere (Durand M, Gallant M A, de Brum-Femandes A J., “Prostaglandin D2 receptors control osteoclastogenesis and the activity of human osteoclasts”. J Bone Miner Res. 2008 July; 23(7):1097-10) FIG. 3 illustrates results of resorption activity studies, where higher resorption area reflects greater resorption OC activity. These results show that the group of patients with RA has higher resorption levels than the control group.

### Example 4

### Diagnosis of RA Severity

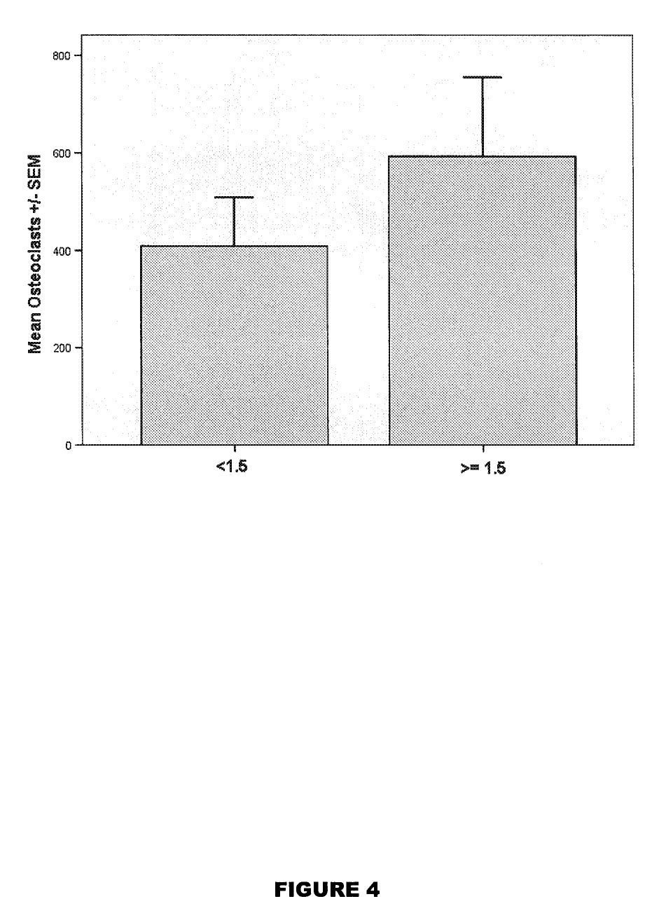

The correlation of RA disease severity to the number of osteoclasts generated in vitro was studied in a cohort of 38 patients randomly chosen from the cohort of 139 RA patients of Table 1. Disease severity was defined as the ratio of the Sharp score of joint destruction (van der Hijde, D., “How to read radiographs according to the Sharp/van der Heijde method”. J Rheumtol 26(3): 743-745, 1999) and the number of years of disease. This gives a value that reflects the speed of progression of RA or severity. The inventors used a cut-off of 1.5, where values equal to or above 1.5 indicate a subject with severe RA, while those below have less severe RA. To determine whether OC values in a patient correlate with severity of RA, PBMC samples were obtained from all 38 subjects, differentiated for 21 days as described above under conditions that promote production of OCs and the mean number of OCs were determined for each group of subjects. As can be seen in FIG. 4, subjects with more severe RA have higher mean OC values than those with less sever RA status. As such, obtaining a blood sample from a subject, culturing the PBMC cells to produce OCs and determine the number of OCs for the subject, as compared to a control or control values (e.g. for subjects without RA or values or value ranges established for patients with known RA severity) would be indicative of severity of RA.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

TABLE 1
Demographics
Table 3. Baseline characteristics of the patients.†

| | Control (Ctl) | RA-total (RAt) | RA-active (RAa) | RA-inactive (RAi) | OA |
|---|---|---|---|---|---|
| Characteristics | (N = 41) | (N = 139) | (N = 77) | (N = 62) | (N = 52) |
| Age -- yr | 57.1 ± 1.2 | 60.8 ± 1.0* | 62.1 ± 1.3* | 59.2 ± 1.5 | 60.8 ± 0.97 |
| Female sex -- no. (%) | 23 (56.1) | 94 (67.6) | 59 (76.6)* | 35 (56.5) | 38 (73.1) |
| Menopause -- no. (%) | | | | | |
| Menopause | NA | 64 (68.1) | 43 (72.9) | 21 (61.8) | 26 (68.4) |
| Pre-menopause | NA | 4 (4.3) | 2 (3.4) | 2 (5.9) | 1 |
| Body-mass index‡ | 27.6 ± 0.9 | 26.6 ± 0.4 | 26.6 ± 0.6 | 26.5 ± 0.6 | 30.8 ± 1.0 |
| Ethnic group - no. (%)¶ | | | | | |
| Caucassian | 38 (92.7) | 133 (95.7) | 72 (93.5) | 61 (98.4) | 52 (100) |
| Other | 3 | 6 | 5 | 1 | 0 |
| Smoking status - no. (%) | | | | | |
| Ever smoke | 15 (36.6) | 84 (60.4)** | 42 (54.5)** | 42 (67.7)** | 27 (51.9) |
| Alcohol status - no. (%) | 32 (78.0) | 67 (48.6)*** | 35 (45.5)** | 32 (51.6)** | 28 (53.8) |

†Plus-minus values are means ± SEM.
CRP denotes C-reative protein,
SR denotes sedimendation rate and
COPD denotes coronary obstructive pulmonary disease.
‡Body-mass index is the weight in kilograms divided by the square of the height in meters.
¶Ethnic group are self-reported.
*P ≦ 0.05 for the comparison between this group and the control group.
**P ≦ 0.01 for the comparison between this group and the control group.
***P ≦ 0.001 for the comparison between this group and the control group.
+ P ≦ 0.05 for the comparison between this group and the RA active group.

What is claimed is:

1. A method for diagnosing, predicting severity of, and monitoring rheumatoid arthritis in a subject comprising assaying in a sample from the subject the number of osteoclasts derived from peripheral blood mononuclear cells and/or osteoclast apoptosis and/or osteoclast resorptive activity wherein an increase in number of osteoclasts and/or a decrease in osteoclast apoptosis and/or an increase in osteoclast resorptive activity compared to a control sample is indicative of rheumatoid arthritis.

2. A method as claimed in claim 1 wherein the number of osteoclasts derived from peripheral blood mononuclear cells is determined and an increase in the number of osteoclasts compared to a control sample is indicative of rheumatoid arthritis.

3. A method as claimed in claim 1 wherein osteoclast apoptosis is assayed and a decrease in osteoclast apoptosis compared to a control sample is indicative of rheumatoid arthritis

4. A method as claimed in claim 1 wherein osteoclast absorptive activity is assayed and an increase in osteoclast resorptive activity compared to a control sample is indicative of rheumatoid arthritis.

5. A method as claimed in claim 1 wherein osteoclast number is assayed and an increase in osteoclast number reflects disease severity and/or progression rates of joint destruction.
